(19) United States
(12) Patent Application Publication
(10) Pub. No.: US 2005/0277597 A1
(43) Pub. Date: Dec. 15, 2005

(54) Use of N-L-alpha-aspartyl-L-phenylalanine 1-methyl ester to treat sunburn and other burns

(75) Inventors: Carl V. Manion, Oklahoma City, OK (US); Allen B. Edmundson, Edmond, OK (US); John E. Adams, Nichols Hills, OK (US); Tony E. Hugli, San Diego, CA (US)

Correspondence Address: CONLEY ROSE, P.C., 5700 GRANITE PARKWAY, SUITE 330, PLANO, TX, 75024, US

(73) Assignee: Oklahoma Medical Research Foundation, Oklahoma City, OK (US)

(21) Appl. No.: 11/147,962

(22) Filed: Jun. 8, 2005

Related U.S. Application Data
(60) Provisional application No. 60/578,716, filed on Jun. 10, 2004.

(51) Int. Cl. A61K 38/04

(52) U.S. Cl. 514/19

(57) ABSTRACT

A method and formulation for treating skin for sunburn and other types of burns is disclosed. The method comprises the topical application of a preparation comprising N-L-alpha-aspartyl-L-phenylalanine 1-methyl ester and its lower alkyl ester derivatives.

### CROSS REFERENCE TO RELATED APPLICATIONS

This application claims priority to U.S. Provisional Application No. 60/578,716 filed Jun. 10, 2004, and entitled “Use of N-L-Alpha-Aspartyl-L-Phenylalanine 1-Methyl Ester to Treat Sunburn and Other Burns, by Carl V. Manion, et al., incorporated herein by reference.

### STATEMENT REGARDING FEDERALLY SPONSORED RESEARCH OR DEVELOPMENT

Not applicable.

### REFERENCE TO A MICROFICHES APPENDIX

Not applicable.

### TECHNICAL FIELD OF INVENTION

This invention concerns the field of treating sunburn and other types of burns.

### BACKGROUND OF THE INVENTION

Upon overexposure to the sun or an artificial source of ultraviolet (“UV”) light, the skin may turn red and, in severe cases, blister. This occurs when the amount of exposure to the sun or other ultraviolet light source exceeds the ability of melanin, a protective pigment in the skin, to protect the skin. It is estimated that a light skinned person may be sunburned in fifteen minutes or less.

The symptoms of sunburn may be red and/or painful skin. There is generally a delay between when the damage has occurred and when it is evident. Pain may occur between six and forty-eight hours after sun exposure, and in severe cases may continue for a longer period of time. In addition to redness and pain, subjects may experience blistering of the skin and a long and debilitating process to return the skin to a healthy state. The damaged skin may peel. Usually peeling starts three to eight days after sun exposure.

Edema (swelling) may accompany sunburn. It has been reported that toxins may be released and fever may accompany the release of toxins.

Long term effects of sunburn are possible skin cancer, skin damage and accelerated aging, age spots, and cataracts.

Chemical burns can also be deleterious to the skin and ocular area. Chemical burns caused by exposure of skin to acidic or basic substances are common in the household and industrially. Currently, such burns are treated by irrigation for decontamination and relief. Severe burns may be followed with additional treatments, and skin grafts may even be necessary.

Thermal burns result from skin exposure to heat or flame. First and second degree burns are normally treatable with wound cleansing, usually daily, and dressing and redressing. Pain medications may also be prescribed.

U.S. Pat. No. 5,654,334 discloses N-L-aspartyl-L-phenyalanine 1-methyl ester (APM) and its derivatives as a pain reliever which is especially effective in relieving pain associated with osteoarthritis and multiple sclerosis. Further, International Application WO 97/00692 discloses APM as an antipyretic.

It has now been found that topical application of N-L-aspartyl-L-phenyalanine 1-methyl ester and its lower alkyl ester derivatives (collectively referred to herein as “APM”) are effective in treating sunburn and other types of burns.

### SUMMARY OF THE INVENTION

In one aspect, the invention is the use of the compound 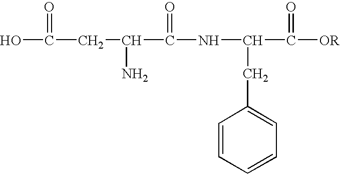
where R is H, CH₃ or an alkyl having 2-4 carbon atoms in a method for treating skin overexposed to sun, heat, or chemicals. A topical preparation may be prepared by adding the compound to a carrier, preferably a lotion or cream, a sunscreen or sunblock preparation, or an osmotic solution such as physiologic saline. A preferred compound has R=CH₃.

### DETAILED DESCRIPTION

Exposure to excess sun, heat or UV causes damage to skin protein or cells which lead to tissue catabolism to eliminate damaged protein as a noxious substance. This process is usually associated with perceived heat, redness, swelling, and may be accompanied by pain as well. It has now been found that topical application of APM to the affected area is an effective treatment method to provide relief from some or all of these symptoms. In addition, APM can be used as an ingredient in a topical sun blocker formulation. While APM has some ultraviolet-light absorbing properties, it also can ameliorate or begin the healing process of skin overexposed to the sun when used in such a prophylactic application to the skin.

Any amount of APM may provide a beneficial reduction in symptoms. A suitable formulation should contain from about 0.1 mg APM per ml of carrier to about 50 mg per ml of carrier. Preferably, a formulation effective for preventing sunburn or reducing symptoms of sunburn or other burns is at least about 1 mg per ml of carrier. Most preferably, a quantity of APM from about 1.2 mg to about 12 mg per ml of carrier is employed in the formulation. The upper limit of APM utilized in the formulation is not critical since the objective is to provide a formulation which may contact the affected area and provide sufficient active ingredient to exert an effect. It is desirable to employ, therefore, the maximum amount of APM per ml so that the resulting formulation has the desired characteristics. In one instance, it may be desired that the APM used be soluble in the formulation, and in another, that the APM used be suspended in the formulation. In the latter instance, body fluid will solubilize the APM from the suspension during use so that the beneficial effects of APM can be realized. In one example, commercially available APM formulated as a powdered sweetener for beverages and foods may be used to formulate a topical preparation. APM powdered sweeteners typically contain about 36.7 mg APM per gram of powder, which may contain dextrose, maltodextrin and aspartame. One gram of powdered sweetener is normally provided in a commercial packet. The packets can be used to formulate a topical preparation by, for example, providing 1 to 8 packets of powdered sweetener per ounce (29.6 ml) of a lotion base. An effective topical APM preparation may be made by mixing a sufficient number of powdered sweetener packets, such as those sold under the brand name EQUAL® (Merisant Co., Monteno, Ill., USA) to a quantity of lotion until the resulting topical preparation has a gritty consistency, but still may be spread on the affected area. Such a preparation which utilizes a lotion base is estimated to contain from about 1.2 mg APM/ml (37 mg APM/fluid ounce) to about 10 mg APM/ml (296 mg APM/fluid ounce).

In a preferred embodiment, a topical formulation is prepared using purified APM in the range of about 0.1 to about 50 mg per ml of carrier. A purified APM is available from SinoSweet Co., Ltd.(Yangzhu, Dapu Township, Yixing, Jiangsu 214226, China) and is characterized as an odorless, crystalline powder, which is chemically pure and contains no additives, preservatives, or colors. A suitable preparation may be made by using about 0.1 to about 5% weight APM/volume of carrier, and most preferably about 1 to 2% weight/volume or 10 to 20 mg/ml of carrier.

In one example, commercially available APM formulated as a powdered sweetener for beverages and foods may be used to formulate a topical preparation for application to an affected area of the skin. APM powdered sweeteners typically contain about 36.7 mg APM per gram of powder, which powder typically contains dextrose, maltodextrin and aspartame. One gram of powdered sweetener is normally provided in a commercial packet. The packets can be used to formulate a topical preparation by, for example, providing 1 to 8 packets of powdered sweetener per ounce (29.6 ml) of a lotion base. An effective topical APM preparation may be made by mixing a sufficient number of powdered sweetener packets, such as those sold under the brand name EQUAL® (Merisant Co., Monteno, Ill., USA) to a quantity of lotion until the resulting topical preparation has a gritty consistency, but still may be spread on the affected area. Such a preparation which utilizes a lotion base is estimated to contain from about 1.2 mg APM/ml (37 mg APM/fluid ounce) to about 10 mg APM/ml (296 mg APM/fluid ounce).

It is contemplated that in the cream, undissolved crystals will be present and will remain undissolved until applied. After application to the burned area, APM will bind to tissue proteins and burn secretions (the fluids that form blisters) and this will further desaturate, dilute and dissolve undissolved crystals in the preparation. The direct binding and dissolution of the available crystals facilitates topical activity and APM penetration. It is contemplated that some degree of gentle debridement may occur because of the particulate APM prior to the dissolution of the crystals. The solubility of aspartame in water is dependent on pH and temperature, the maximum solubility is reached at pH 2.2 (20 mg/ml at 25° C.) and the minimum solubility at pH 5.2 (pHi) is 13.5 mg/ml at 25° C. The stability of aspartame is dependent on time, temperature, pH and water activity. For use in the present invention, a solution is desired at 25° C., pH 7.4. In order to achieve maximum solubility, of asparatame at this temperature and pH, one should dissolve APM or its derivative in a solvent such as sterile water or physiological saline. Preferably, the solution should be made up fresh since APM is more stable in the dry state. At a pH greater than 5, cyclisation with the formation of diketopiperazine reportedly occurs in APM solutions. Alternatively, a stable solution of APM at room temperature, pH 4.3 can be made and the pH adjusted to 7.4 just prior to use. In addition, a solution of APM at pH 7.4 can be made and frozen so that it can be thawed just prior to use. In order to sterilize a solution for which sterility may be desired (e.g. application to the eye or to an open wound), one may use a sterilizing technique that will not be harmful to the molecule. Since heat may degrade APM and its derivatives, one may use a sterilizing filter or other methods that will not harm the APM or derivative. It is acceptable to apply a solution which is saturated with APM and in which crystalline materials are present. Such a solution may assist with debridement, or the gentle removal of dead and dying cells. Since the solution is not intended for IV use, the presence of crystalline material will identify its intended topical use. The absence of crystals would identify that the solution has reached the limits of its life because of solubility considerations.

In a preferred method for making an APM solution for use at physiological pH, the APM is dissolved in sterile water and passed through a sterilizing filter, such as a HEPA (high efficiency particulate absorber) filter capable of excluding particulate matter one micron or larger, into a sterile container. The sterile container may be subjected to lyophilization, and the lyophilized APM stored until ready to use in a sealed, sterile container such as commonly used in the pharmaceutical industry. The lyophilized APM may be added to either a smaller or larger quantity of prepared sterile water or saline to either dissolve completely or to create a saturated solution. For example, a quantity of 250 mg APM can be added to a sufficient amount of sterile saline to achieve a concentration of 50 mg/ml, and in this case the solution will contain crystals. Alternatively, 250 mg APM can be dissolved in a larger volume of sterile saline. Using the lyophilized APM will avoid the pH and temperature degradation of APM at the desired pH 7.4 for use in the eye and open wounds. APM can dissolve in carriers other than water to increase its solubility. It has been found that for most purposes, sufficient APM will dissolve to provide an effective product as described above. However, the limitations of solubility and pH for water can be avoided by using a cream or ointment or a compatible solvent in which APM has increased solubility. One may employ a skin-compatible organic solvent in the aqueous solution to increase solubility. The other advantage of an organic solvent is that it may help transfer the material into the skin. For example, addition of alcohols such as ethanol or propanol enhance the solubility of APM in an aqueous mixture. The pH range for a topical preparation is generally from pH 4.0-4.5 (skin pH) to pH 7.4 (physiologic blood pH). The topical products are appropriate for topical use at a pH near the pH of the skin (about pH 4.5). If it is desired to use the APM preparation on broken skin, it is desirable to raise the pH to approach physiologic pH so that the acidity will not be uncomfortable.

The methodology for making APM is known in the art, and APM is available commercially. Its preparation is disclosed in U.S. Pat. No. 3,492,131. It is believed that various modifications can be made to the APM molecule, and the resulting derivatives will also have utility in the claimed invention. Since the 1-methyl ester portion of the molecule is not believed to contribute to the therapeutic activity of the molecule, N-L-alpha-aspartyl-L-phenylalanine itself or other lower alkyl esters are believed to be effective. Other possible analgesic physiologically acceptable derivatives are believed to include N-acyl-L-(beta-substituted)-aspartyl-L-phenylalanine lower alkyl esters and N-acyl-L-(beta-substituted)-aspartyl-L-phenylalanine. Chemical modifications made to the APM molecule which do not reduce the physiologically active properties disclosed herein thus fall within the scope of this invention

### EXAMPLE 1

### Use of an Aspartame-Containing Cream to Treat Sunburned, Heat or UV Damaged Skin

A person with sunburn, heat or UV damaged skin may apply a layer of a cream or lotion containing 250 mg of APM per ounce of a cream or lotion base twice per day or as needed to relieve redness, swelling or pain.

### EXAMPLE 2

### Use of APM-Containing Composition to Prevent Sunburn

A cream may be prepared which can be used to prevent sunburn by application of the cream to the skin in advance of sun exposure or any undesired ultraviolet (“UV”) light exposure. In this case, APM at 1 to about 50 mg/ml APM, or preferably 1.2 to 10 mg/ml can be added to a sun blocker formulation and serves to helps to ameliorate a sunburn and/or begin to heal a sunburn that may occur even though the sun blocker formulation may contain sunblocker active ingredients such as, but not limited to, para amino benzoates (“PABA”), salicylates, cinnamates, anthranilate, camphors (all UVB absorbers), benzophenones (UVB and UVA absorber), dibenzoylmethanes (UVA absorber) or other ingredients. Such a formulation may also be useful in treatment of overexposure to sun.

### EXAMPLE 3

### Use of Aspartame to Treat Chemical Burns

Skin damage or burns may be caused by chemicals, and a subject may experience swelling, redness, and pain, similar to the symptoms experienced by a sunburned individual. A person with chemical or irritant burns may wash the affected area with a wash solution containing APM in a solution at pH 7.4. A layer of a pharmaceutically suitable cream as described in Example 1 may be applied after washing with an APM solution or with a different solution such as water or saline, and such application may reduce inflammatory symptoms and pain.

### EXAMPLE 4

### Using Aspartame to Treat Active Burns

A person with blistered and burned skin areas may apply a physiological sterile saline solution containing the maximum soluble amount of APM at pH 7.4, 25° C. In addition, such a solution may be applied to nasal and/or ophthalmic mucosa. The suggested treatment regimen is to apply the solution to the affected area. The bum area should be kept moist. The solution may be poured on the affected area, the treated area covered with a semiocclusive dressing such as a gauze or special non-stick dressing and kept moist with the sterile solution by applying twice daily or as needed.

If appropriate, an APM formulation may be prepared by using other pharmaceutical bases such as petrolatum, aloe vera, cocoa butter containing oils in the range provided herein. Topical application of APM containing cream to the affected skin areas should effect accelerated healing with decreased swelling and pain.

1. A method for treating skin burns, comprising topical application of a preparation comprising APM and a pharmaceutical carrier to a subject in need of such treatment.

2. The method of claim 1, wherein said preparation comprises from about 0.1 to about 50 mg APM/ml.

3. The method of claim 1, wherein said preparation comprises from about 1.2 mg APM/ml to about 10 mg APM/ml.

4. The method of claims 1-3, wherein said pharmaceutical carrier is at physiological pH.

5. The method of claims 1-3 wherein said preparation is from about 5.4 to about 7.4 pH.

6. The method of claims 1-3, wherein said fluid is physiological saline.

7. The method of claims 1-3 wherein said preparation is sterile.

8. The method of claim 4 wherein said preparation is sterile

9. The method of claim 5 wherein said preparation is sterile.

10. The method of claim 6, wherein said preparation is sterile.

11. The method of claim 1, wherein said burn is sunburn.

12. The method of claim 1, wherein said burn is chemical.

13. The method of claim 1, wherein said burn is thermal.

14. The method of any of claims 1-3, wherein said preparation is applied two or more times per day.

15. A method for preventing damage to the skin caused by overexposure to sun or a UV source, comprising applying a formulation comprising APM to the skin prior to or during exposure to said sun or UV source.

16. The method of claim 15, wherein said preparation is applied two or more times per day.

17. The method of claims 15 or 16, wherein said formulation contains between about 1 and about 50 mg/ml APM.

18. A formulation for use in preventing damage to the skin from overexposure to sun or a UV source, comprising APM and a chemical blocker of UV light.

19. A sterile solution comprising APM at pH 5.4-pH 7.4.

20. The solution of claim 19, wherein said solution contains APM at the maximum solubility.

21. The solution of claim 20, in lyophilized form.

22. A formulation for topical application comprising APM in a carrier at a concentration or from about 0.1 mg to about 50 mg APM per ml of carrier.
